(19) United States
(12) Patent Application Publication
(10) Pub. No.: US 2019/0091423 A1
(43) Pub. Date: Mar. 28, 2019

(54) Hot Oil Tank

(71) Applicant: Brett William Tygett, Folsom, CA (US)

(72) Inventor: Brett William Tygett, Folsom, CA (US)

(21) Appl. No.: 15/713,701

(22) Filed: Sep. 25, 2017

(51) Int. Cl. A61M 15/06; A61M 11/04; A61M 15/00; A24F 47/00; A61K 36/185; A61K 9/00

(52) U.S. Cl. CPC A61M 15/06; A61M 11/042; A61M 15/0021; A61M 2205/8206; A61K 36/185; A61K 9/007; A24F 47/008

(57) ABSTRACT

Sole purpose of this invention process is to discretely and freely gravity feed highly viscous and solid matter medical marijuana concentrate substances from within a portable, closed and compact vaporizer system, offering protection of the very costly chambered concentrates as well as allow the user to abide by or use discretion with the use of their medicinal concentrate substance.

### BACKGROUND OF THE INVENTION

The medical marijuana industry has allowed for new manners of producing concentrated marijuana substances yet the ability to process the thick, viscous concentrate oils in a vaporizer cartridge similar to E-Cig does not exist, processes only exists for vaporizing thinned oils or low viscosity substances; this process has made an exception and will allow for processing of these thick and sticky substances. Art included in pictures shares the process which has no flaws or limitations known to exist.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE OR COMPUTER PROGRAM LISTING COMPACT DISC APPENDIX

Not Applicable

### BRIEF SUMMARY OF THE INVENTION

Vaporize Viscous Concentrate Marijuana Oils in adapted vaporizer module.

### DESCRIPTION OF DRAWING VIEWS

FIG. 1A Perspective view showing unmodified traditional vaporizer cartridge glass chamber concentrate containment unit

FIG. 1B View showing unmodified traditional vaporizer center screw connector

FIG. 1C View showing unmodified traditional vaporizer atomizer section

FIG. 1D View showing unmodified traditional vaporizer base connector

FIG. 2A View showing how the material heating element will sit in FIG. 1A

FIG. 2B View showing the modification to FIG. 1B

FIG. 2C Close-up view of reference 1G from FIG. 1C

FIG. 2D View showing modification to FIG. 1D

FIG. 3 Example of how the electrical flow could work

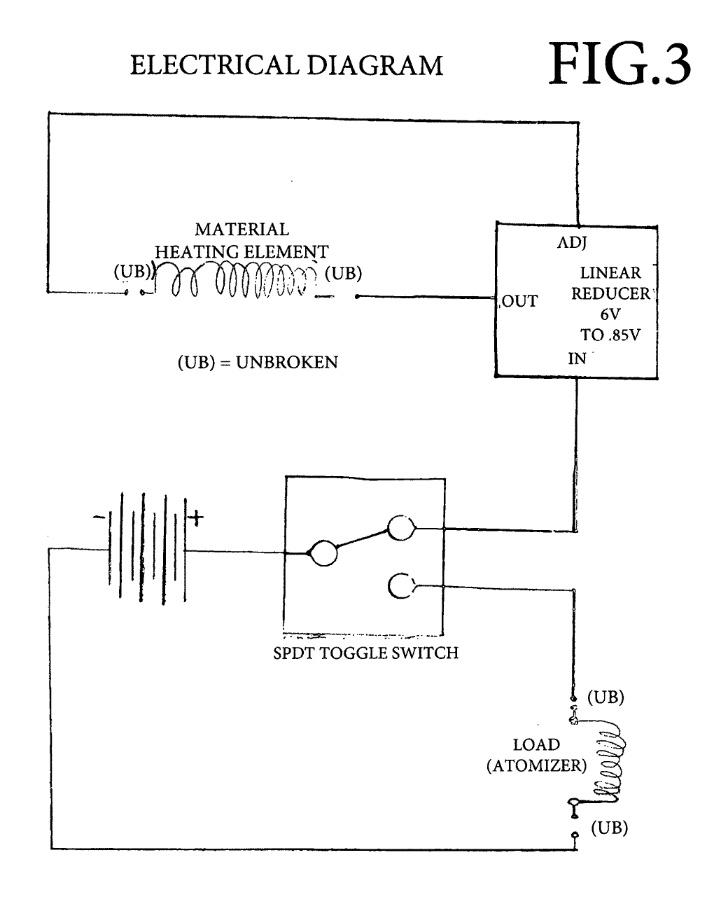

### DESCRIPTION OF REFERENCE CHARACTERS

1—Original Design (could be any Vaporizer Cartridge)

1A—Traditional Mouthpiece

1B—Traditional Container

1C—Traditional Center Screw Connector

1D—Traditional Screw Receiver

1E—Traditional Rubber Gasket

1F—Traditional Atomizer Top

1G—Traditional Atomizer+Coil

1H—Traditional Atomizer insulator gasket.

1I—Traditional Atomizer Base

1J—External battery (as ‘Example’ of power source)

### 2—Shows Specific Embodiment Changes to Vaporizer Cartridge

2A—Stainless Steel heating intra filament coated in a non-conductive, non-corrosive material . . . To heat the viscous oil to liquid state and allow unimpeded flow.

2B—Filament Ends

2C—Hole added to allow the filament to meet its power source.

2D—Insulator Grommet. To allow safe pass-through for the wire and prevent leakage of material

2E—Wire Connector. Female (To 2F).

2F—Wire Connector. Male (To 2E).

2G—Linear Voltage Regulator (mcp1702-150 3-pin). To regulate voltage and keep a low temperature on “2A”.

2H—Single-Pole, Double-Throw Switch. On-On preferred, though On-Off-On is acceptable.

2I—Holes for wire from (2J) to atomizer coil and the battery.

2J—Coil wire connects to battery to make complete circuit (2H)

### DETAILED DESCRIPTION OF THE INVENTION

Discretely vaporize ‘medical marijuana concentrate substances’, referred to as hemp/cannabis/CO2/Hash and etcetera marijuana oil, in a closed and air tight/liquid tight/leak-proof and sealed chamber in a MODIFIED traditional vaporizer cartridge with the concentrate substance in its purest form, in the chemical state of being a high viscosity CO2 style hemp oil/gel with a gluiness/tacky texture similar to the consistency of Heinz Ketchup (or more viscous), with a VISCOSITY (centipoises) of 50,000-70,000 (highly viscous), and in comparison—Water, for example, being a 1-5 (Low) VISCOSITY at Room Temperature http://www.cstsales.com/viscosity.html . . . E-Cigarette Juice is averaging 300 Viscosity http://kv5r.com/vape-liquid-viscosity/ . . . This process also will allow the option of a solid CO2 OR OTHER extraction concentrate method referred to as Hash/BHO/Shatter/etcetera, a solid, hard candy-like consistency https://medium.com/meetharmony/cannabis-extractions-the-complete-guide-151edb382 d65

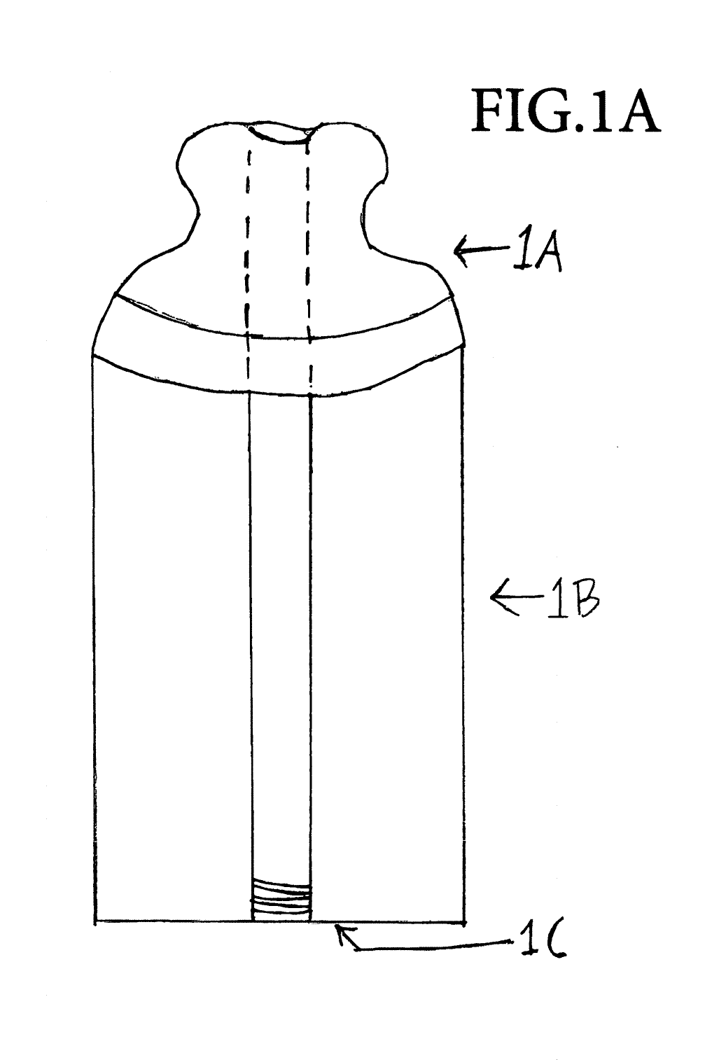

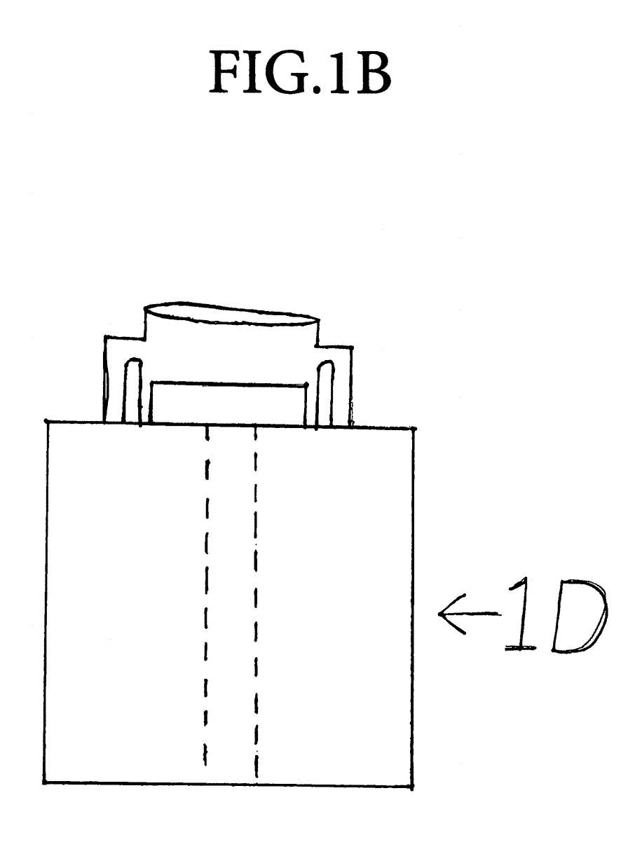

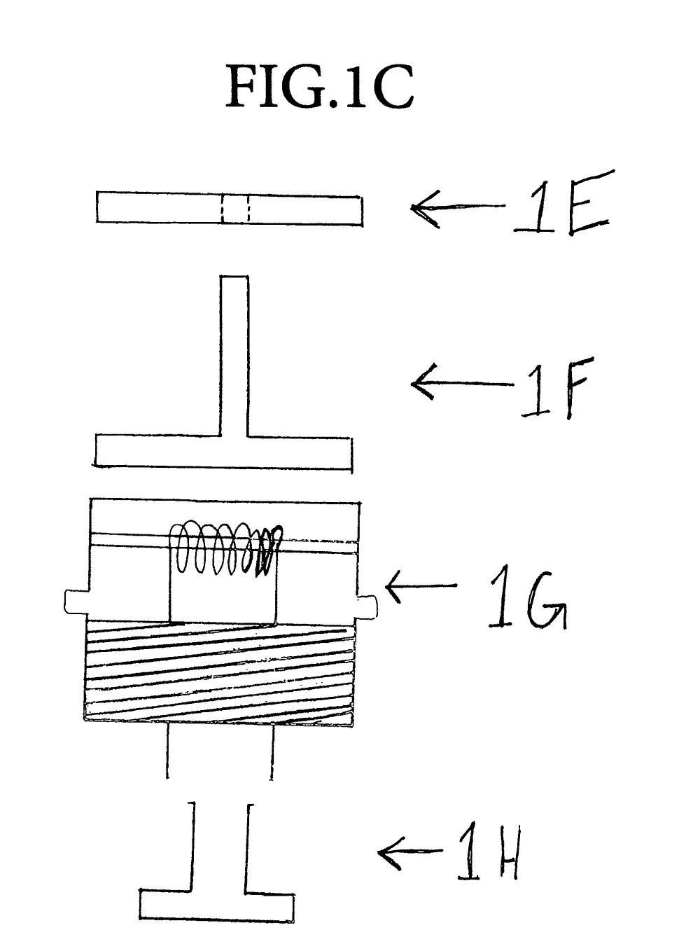

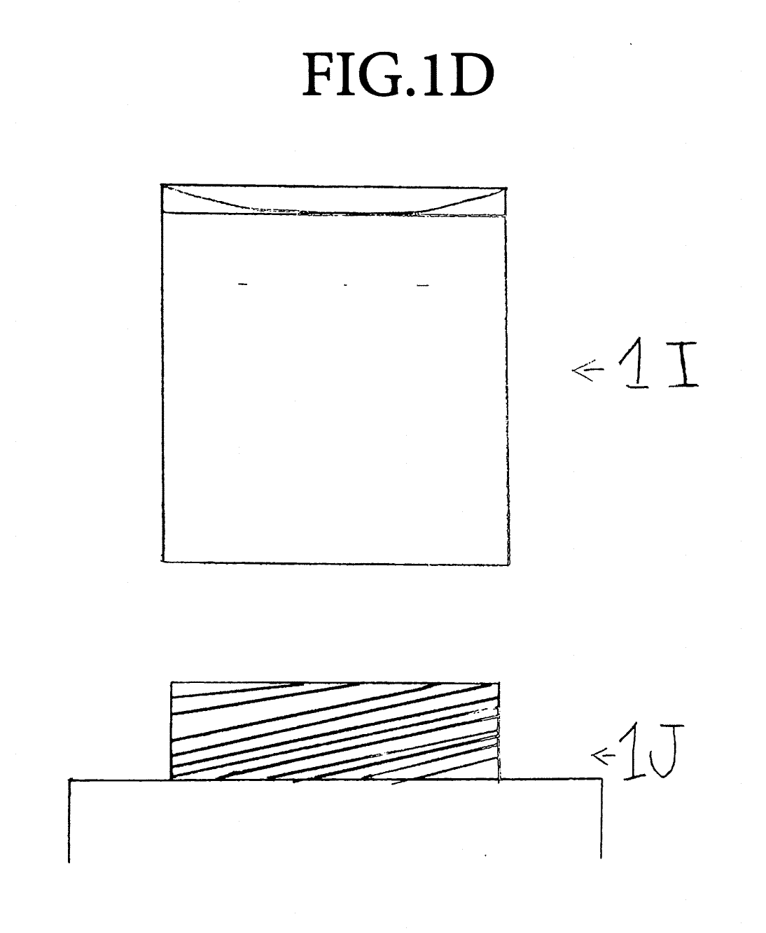

FIG. 1A Illustrates a traditional vaporizer with . . . 1A mouth piece, fed by 1C center screw connector and vapor pass through to mouthpiece . . . 1B shows the glass chamber for contents of cartridge to be warmed in the cartridge chamber and gravity fed through FIG. 1B, 1D the bottom of the cartridge

FIG. 1C, 1G shows the point of contact in the vaporizer that allows the product to meet the atomizer coil with other assembly components, 1E traditional rubber gasket, 1F traditional atomizer top, 1H traditional atomizer insulator gasket.

FIG. 1D, 1I is the lower most cartridge base which attaches to 1J external battery unit shown and once all of the aforementioned are connected, this then forms the traditional vaporizer cartridge.

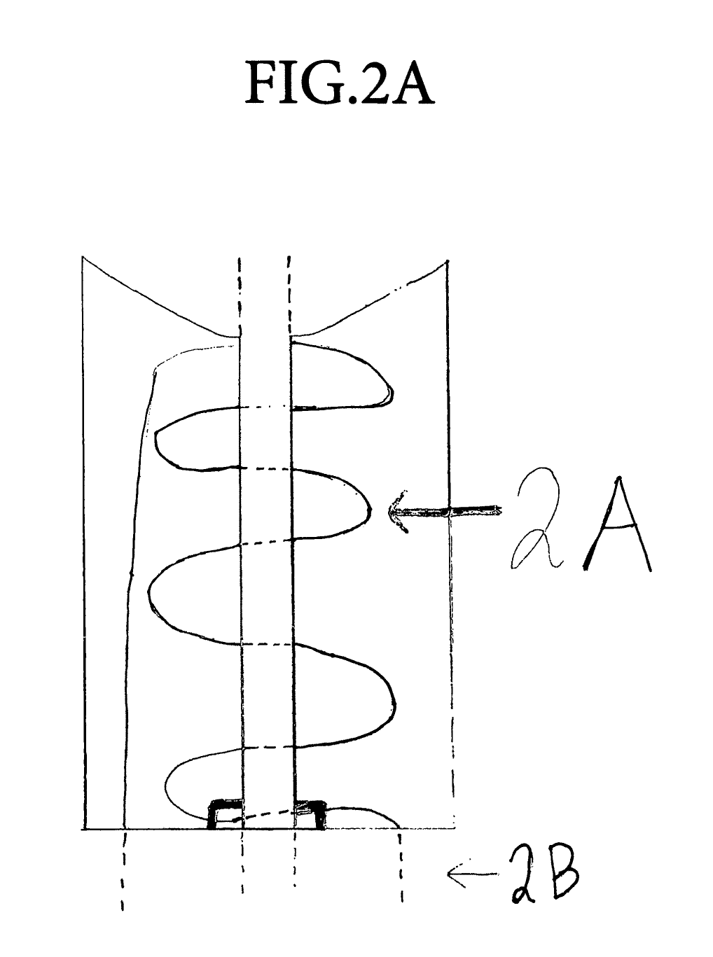

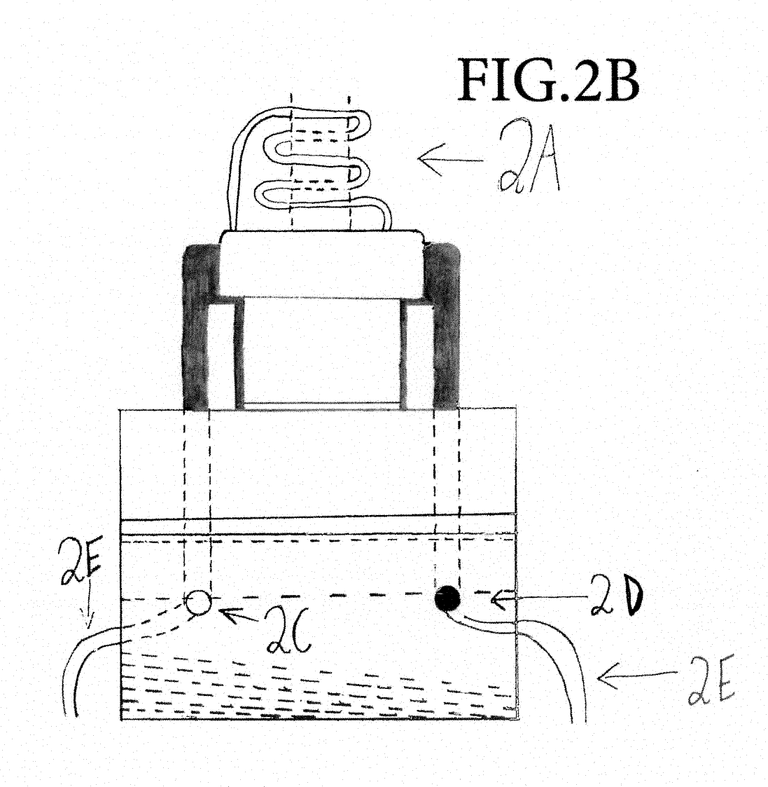

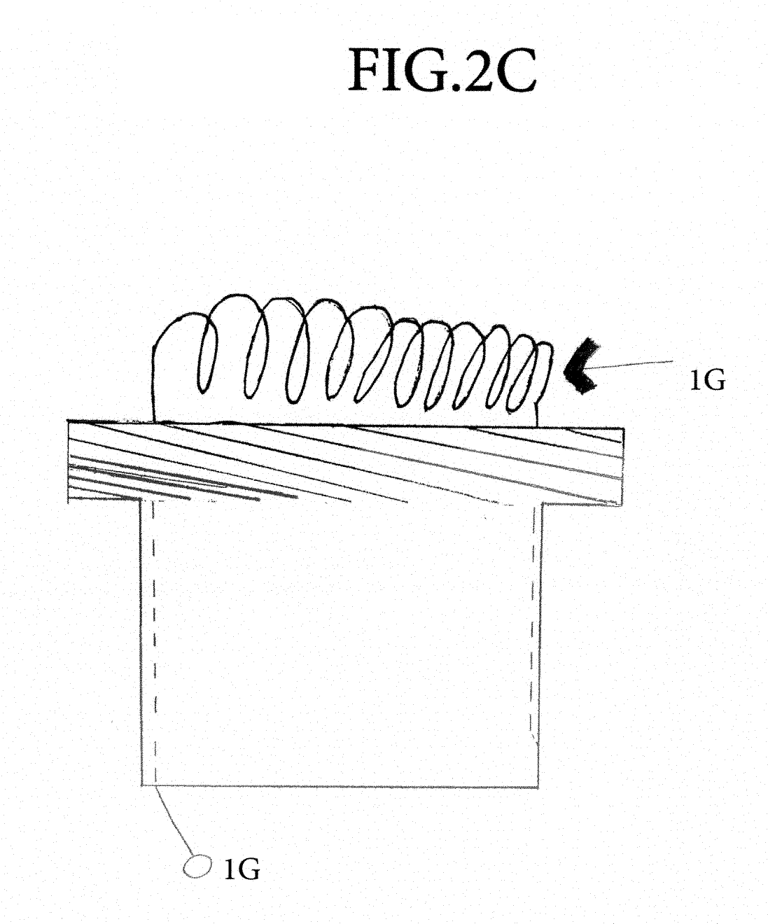

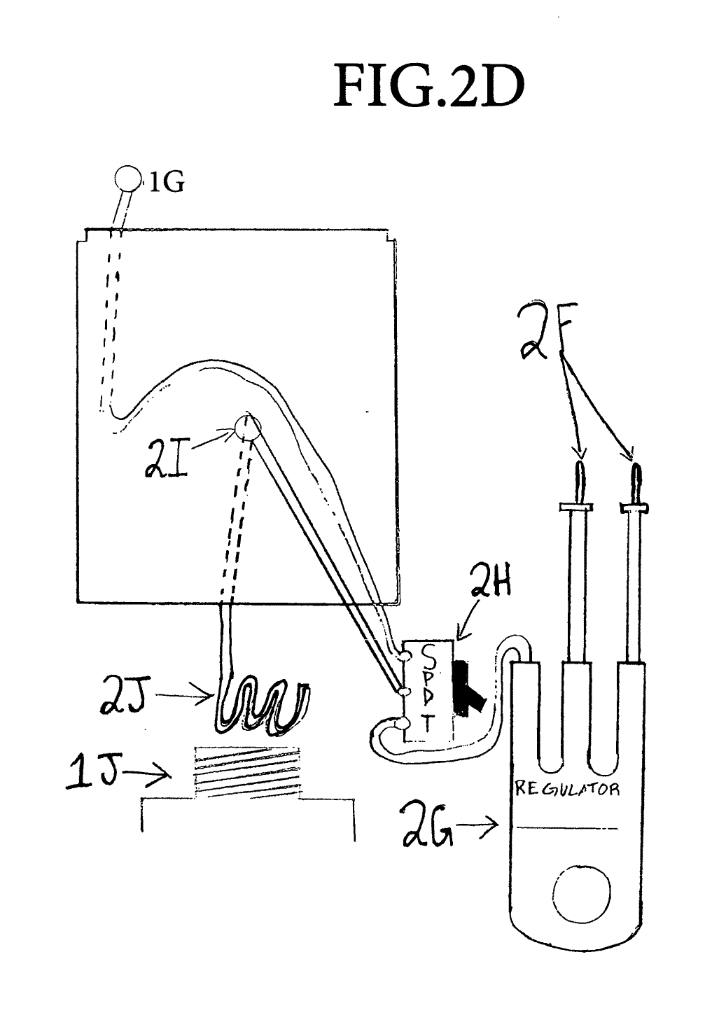

FIG. 2A, 2A Stainless steel heating intra-filament coated in non-conductive, no-corrosive polymer that is modified to allow a switch to direct a reduced current to warm the concentrate substance via the ‘intra-filament winding’ inside the cartridge container, which will then make contact and be in proximity to the concentrate allowing the substance to be warmed, 2b is the filament ends.

FIG. 2B illustrates the previous FIG. 1A, FIG. 1B, FIG. 1C and FIG. 1D, 1I now fully assembled with 2C holes shown in FIG. 1D, 1I allowing passage through 2D grommet and 2E shows the filament end wires which connect by incorporating

FIG. 2C 1G atomizer coil wires wire ends

FIG. 2D, 2I atomizer coil wire holes, one to feed 2J wire end to the battery 1J with the other 1G circuit wire connecting to 2H an SPDT toggle switch and when 2H is changed, a lead to 2G powers 2F which will connect back to FIG. 2A, 2B filament wire ends where the now reduced current will warm the concentrate substance via FIG. 2A, 2A the ‘intra-filament winding’ inside FIG. 1A, 1B the cartridge container, which will then make contact and be in proximity to the concentrate allowing the substance to be warmed, then the warmed and now low viscosity substance is free to gravity feed past FIG. 1B, 1D inclusive of a cotton, fiberglass or silica type barrier, gravitating and making contact with FIG. 1C, 1G the traditional metal coil atomizer heating element (powered from FIG. 1D, 1J a Separate [external] Battery Unit) . . . the special characteristic of this process is a Modified (Traditional) Vaporizer Cartridge that is switchable FIG. 2D, 2H to ‘heating/warming’ the FIG. 1A, 1B sealed and closed cartridge concentrate contents, FIG. 2D, 2H a push button switch allows the cartridge to switch from the warming filament and to FIG. 1C, 1G the traditional metal coil atomizer heating element which will vaporize a controlled amount of the concentrate substance delivered to FIG. 1A, 1A the cartridge mouthpiece once concentrate can be delivered to the coil atomizer, with power derived from FIG. 1D, 1J a Separate/External Battery Unit. See diagrams of art attached

Different concentrates will heat and flow though at different heat rates (heating times), due to the nature of the state of matter of the substance being different from batch to batch or differing in consistency from manufacturer to manufacturer. A user will learn what time allotment is needed to warm the substance for a pleasurable experience, a process currently unavailable unless one uses a glass bong apparatus with an electrically heated, red hot tip or torch heated tip for smoking the concentrate substance off of the heated tip, referred to as the ‘nail’ . . . or, in my case, I heat my vape cartridge in the front of a floor heater until the substance passes to the atomizer; not very conducive in the summertime while indoors.

1. This method is threefold, a three step process: The embodiment of this process of discretely heating the very costly and highly viscous medical marijuana concentrates for free-flow gravity fed vaporizing as a reduced current warms an intra-filament wire, in the cartridge, encapsulated in a traditional handheld and closed vaporizer cartridge atop an external battery unit which allows the user to switch from the vaporizer coil to a direct warming contact and radiant heating contact of the concentrate material in the cartridge allowing for change in the state of matter from a solid to a liquid, allowing better control of dosage for the user; otherwise not attainable with other products on the market because this process allows for all of the chambered material to be fully processed out of the chamber at adjustable temperatures, utilizing all of the matter encapsulated for use.
Firstly a push button switch toggles the voltage current by pressing the battery button to find a lit LED on the switch button or a light in the circuitry will indicate the toggle connection to a linear reducer/voltage reducer, secondly that user holds the battery button to request a current that is linked to the intra-filament winding heating element located inside the cartridge filed with the high viscosity hemp oil/hemp concentrate in a solid state of matter, after a few second burst to visibly warm the concentrate substance, the substance then will gravity feed to the traditional vaporizer coil, as the concentrate is now in the form of a low viscosity liquid, the user will then push the toggle button again to change current (and terminate the LED) to the traditional vaporizer coil atomizer element and receive the vapor at the mouthpiece. Then repeat warming if necessary. Perhaps one warming session is all that would be needed as the user may very well receive the two or three vapor draws as they prefer and be done with their vaporizing session.
Sole purpose of this invention process is to discretely and freely gravity feed highly viscous and solid matter medical marijuana concentrate substances from within a portable, closed chamber and compact vaporizer system, offering protection of the very costly chambered concentrates as well as allow the user to abide by or use discretion with the use of their medicinal concentrate substance.
This is a new process ‘Warming of the Vaporizer Cartridge Concentrate Contents’ that can switch back to vaporizer setting in vaporizer cartridge' to then control the amount of product desired on the atomizer coil for processing. Conventionally, this process is built atop a traditional e-Cig liquid vaporizer cartridge platform and external vaporizer battery technology. An electric current Linear Regulator (a Voltage Regulator) is interrupted or electrified by a push button or toggle switch located on the vaporizer cartridge that allows a controllable reduced current to pass to a warming intra-filament encapsulated in the traditional vaporizer cartridge . . . once the contents warm to a free flowing liquid state the concentrate substance can then pass through a cotton or fibrous barrier to find the metal coil atomizer, a push button switch moves the voltage current back to the ‘traditional’ electronic metal coil atomizer that performs the vaporizing function. This idea, the process of heating the contents is entirely built upon the existing E-Cig Vaporizer Cartridge. I intend to collaborate with existing cartridge manufacturers to have them arrange to manufacture the ‘Hot Oil Tank’ idea, the example cartridge I prefer to use currently is an Atmos Pro T Mini V2, due to its reliability and leak proof qualities (I was advised that the patent on it may be for sale if I desired to inquire). By incorporating a metal intra-filament, encapsulated in the glass or polymer container of the cartridge and adding a push button switch in their existing cartridge designs will easily modify the existing E-Cig cartridge, currently designed to process ultra low viscosity fluids, to a new design that will allow gravity free flow of the concentrate substance to make the passage to the vaporizing coil atomizer a possibility.
